(19) United States
(12) Patent Application Publication
(10) Pub. No.: US 2008/0195047 A1
(43) Pub. Date: Aug. 14, 2008

(54) ENTERAL FEEDING SYSTEMS, DEVICES AND METHODS

(76) Inventor: Mitchell Price, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/028,631

(22) Filed: Feb. 8, 2008

Related U.S. Application Data
(60) Provisional application No. 60/900,649, filed on Feb. 9, 2007.

(51) Int. Cl. A61M 1/00

(52) U.S. Cl. 604/151

(57) ABSTRACT

Systems, devices and methods for enteral feeding that, in some arrangements, are powered by energy produced by a user of the system. Other arrangements utilize an external source of power. Generally, the systems include a storage assembly configured to hold a nutritional substance to be delivered to the gastrointestinal system of a user, such as through a feeding tube. The systems may include a pump unit, which can be manual, mechanical, hydraulic or electric. In some arrangements, the pump unit is separate from the storage assembly.

### RELATED APPLICATIONS

This application is related to, and claims the benefit of, Provisional Patent Application No. 60/900,649, filed Feb. 9, 2007 and entitled, ENTERAL FEEDING PUMP SYSTEM AND METHOD FOR USE, which is hereby incorporated by reference herein in its entirety and made a part of the present disclosure.

### BACKGROUND OF THE INVENTION

1. Field of the Invention

The present inventions generally relate to diet and nutrition. More specifically, the present inventions relate to devices, systems, and methods for enteral or tube feeding and to assisted delivery of a nutritional substance to the gastrointestinal (G.I.) system.

2. Description of the Related Art

Systems, devices, and methods currently exist for providing nutrients to a patient who is unable to ingest food normally. Such systems, devices and methods generally fall within one of the broad categories of intravenous (IV) delivery or enteral delivery. Due at least to the differences in the patient or user and the conditions under which each category of feeding is utilized, the systems, devices and methods related to each of the two broad categories of nutrient delivery are subject to unique constraints and have generally developed along separate paths.

With respect to IV delivery, patients may be treated in a hospital-like setting, or a similar controlled setting. In such settings, patients in need of IV delivery of nutrition are often relatively immobile. Thus, it is feasible in such settings to suspend a bag of nutrients in a liquid form above the patient to utilize gravitational force to dispense the nutrients. Such a method is relatively convenient for the staff and patient, and is cost-effective. However, for mobile or long-term patients or users, IV delivery is inconvenient and is likely to be unsatisfying for the patient or user. Another IV delivery method is total parenteral nutrition (TPN), which in some cases may involve nutrition delivered intravenously by an ambulatory infusion pump. Although such a method permits mobility, it also requires sterile conditions. One possible complication from extended TPN is bacterial infection due to the extended placement of an intravenous catheter used to deliver the nutritional substance.

Enteral feeding methods are often utilized for more long-term nutritional delivery needs, or for mobile users. For example, enteral feeding may be utilized for persons or animals that are unable to ingest food orally, or are unable to ingest enough food orally to meet daily nutritional demands. Possible causes for the inability to meet nutritional needs through oral ingestion of food may include: severe food allergies, head trauma, disease or abnormalities of the gastrointestinal tract, central nervous system problems and metabolic disorders, among others.

In general, enteral feeding, or tube feeding, involves placing a first end of a tube at some point within the gastrointestinal system and leaving a second end of the tube externally accessible. Some common feeding tube placements include passing the tube through the nose and positioning the internal end within the stomach (nasogastric) or other parts of the GI system (e.g., nasoduodenal or nasojejunal), passing the tube through the skin and positioning the internal end within the stomach (percutaneous endoscopic gastronomy—PEG), and passing the tube through the skin and positioning the internal end within the small intestine (percutaneous endoscopic jejunostomy—PEJ). Other arrangements are possible, as will be appreciated by one of skill in the art.

Typically, an extension set is coupled to the feeding tube at a first end and includes a feeding and/or medication port at a second end. Several types of devices may be used to deliver the desired nutritional substance (e.g., milk or formula) to the extension set and, thus, to the feeding tube. For example, a large syringe or a bag (similar to an IV bag) utilizing gravity or even a pump may be used. Each of these existing methods suffers from disadvantages. For example, the syringe method requires the user to mix the nutritional substance in a separate container, and draw the nutritional substance into the syringe. The need to carry an additional storage and/or mixing container, and the need to transfer the nutritional substance from the container to the syringe, makes the syringe method rather inconvenient. Moreover, the user is required to apply a significant force to the syringe plunger. In addition, forces transferred to the feeding tube from the handling and actuation of the plunger may cause discomfort. Further, avoiding the transfer of forces to the feeding tube may require a person to assist the user/recipient of the nutritional substance.

As mentioned above, the bag method may utilize gravity to dispense the nutritional substance. As a result, the delivery rate is determined by gravity and is relatively constant. Accordingly, the user has little to no influence on the flow rate. The user may squeeze the bag to increase the flow rate, but this requires the application of a significant force on the part of the user. In addition, the bag must be maintained above the point of delivery, which reduces the convenience to the user. Furthermore, as the nutritional substance is delivered to the stomach, the pressure within the stomach increases. The increased pressure slows, and can even stop, the delivery of nutritional substance from the bag.

A pump, such as a peristaltic pump, for example, may be utilized to urge the nutritional substance from the bag. In one arrangement, an exit flow tube of the bag is received within the pump and the pump progressively squeezes and releases consecutive portions of a portion of the tubing to pump fluid within the tubing. Such pumps increase the feed rate to decrease the feed time for a given volume in comparison to gravity feeding. In addition, the feed rate may be variable. However, the range of adjustment of the feed rate is typically small and the pumps are expensive. Furthermore, the pumps are often noisy, which inhibits the discreet use of the pump. In addition, it may often be inconvenient to transport the pump due to the size and weight of a typical pump.

### SUMMARY OF THE INVENTION

An aspect of the present invention involves the realization by the present inventor that a significant drawback of all of the prior art methods described above is that the methods do not allow the delivery of the nutritional substance to mimic the range of delivery rates available through oral ingestion of food. One or more of the preferred embodiments of the present invention provide systems, devices and methods of enteral feeding that allow a user to select a desired delivery rate from a wide variety of possible delivery rates, and provide the flexibility to alter the delivery rate, sometimes drastically, during a single feeding session. Such systems, devices and methods allow the user to satisfy hunger demands in a manner similar to oral ingestion.

In addition, one or more preferred embodiments are relatively inexpensive to manufacture and to use. In some arrangements, the more complicated portions of the system or device are reusable for a significant number of feedings. Other portions of the system or device may be disposable, and preferably are simple in construction so as to be relatively inexpensive to manufacture.

A further aspect of the present disclosure and preferred embodiments is systems, devices and methods for permitting the creation of a custom experience based on an individual user's nutritional substance delivery preferences at a given time and in a given place. One or more of the preferred embodiments relate to systems, devices, and methods for flexibly and efficiently delivering sustenance in a matter optimized for enteral feeding. Further provided by the one or more of the preferred embodiments are systems or devices that are easy to assemble, use and easy to clean. Some embodiments are especially well-suited to permit mixing of individual ingredients of a nutritional substance within the feeding device.

A preferred embodiment involves a method of delivering a flowable nutritional substance to a feeding tube. The method includes applying a manual force to an input of a manual pump, pressurizing a pressure chamber in response to energy originating from the application of the manual force to the input of the manual pump, expelling the flowable nutritional substance from a storage chamber in response to the pressure in the pressure chamber, and delivering the expelled flowable nutritional substance to the feeding tube.

Another preferred embodiment involves an assembly for delivering a flowable nutritional substance to a feeding tube. The assembly includes a container having an open end and a substantially closed end, the closed end incorporating a one-way valve that permits a pressurizing fluid to enter the container. A bladder has an open end and is positionable within the container with the open end of the bladder proximate the open end of the container. A lid is securable to the container. The lid and the container are configured to capture the open end of the bladder between the lid and the container to create a pressure chamber between the container and an outer surface of the bladder, and to create a storage chamber between the lid and an inner surface of the bladder. The storage chamber is configured to receive the nutritional substance. A pump is configured to pump the pressurizing fluid into the pressure chamber to cause the nutritional substance to be expelled from the storage chamber.

Yet another preferred embodiment involves an assembly for delivering a flowable nutritional substance to a feeding tube, including a storage assembly for the nutritional substance. The storage assembly includes a container incorporating a valve that permits a pressurizing fluid to enter the container. A lid closes the container and a bladder is positionable within the container. The bladder at least partially defines a storage chamber for the nutritional substance. A pressure chamber is defined between the container and an outer surface of the bladder. A pump unit includes a pump. The pump unit is separate from the storage assembly and is configured to receive the container and to pump the pressurizing fluid through the valve into the pressure chamber to cause the nutritional substance to be expelled from the storage chamber.

### BRIEF DESCRIPTION OF THE DRAWINGS

These and other features, aspects and advantages of the present invention are described below with reference to preferred embodiments, which are intended to illustrate, but not to limit the present invention. The drawings contain nine (9) figures.

FIG. 1 illustrates a person using a preferred embodiment of the present enteral feeding system to deliver a nutritional substance to a feeding tube.

FIG. 2 is a perspective view of the enteral feeding system of FIG. 1. The enteral feeding system includes a storage assembly for the nutritional substance and a pump unit.

FIG. 3 is a cross-sectional view of the enteral feeding system of FIGS. 1 and 2. In FIG. 3, the storage assembly is coupled to the pump unit, which incorporates a manual pump having one or more user input buttons.

FIG. 4 is an enlarged cross-sectional view of a lid and upper portion of a container of the storage assembly of FIGS. 1-3.

FIG. 5 is an enlarged cross-section view of the pump unit and a lower portion of the container of the enteral feeding system of FIGS. 1-3.

FIG. 6 is a flowchart of one possible method of using the enteral feeding system of FIGS. 1-3.

FIG. 7 is a perspective view of a modification of the enteral feeding system of FIGS. 1-3. The system of FIG. 5 utilizes an alternative pump unit incorporating a manual pump actuated by a hand bulb.

FIG. 8 is a perspective view of another modification of the enteral feeding system of FIGS. 1-3. The system of FIG. 6 utilizes an alternative pump unit incorporating a battery powered electric pump.

FIG. 9 is a perspective view of yet another modification of the enteral feeding system of FIGS. 1-3. The system of FIG. 7 utilizes an alternative pump unit incorporating a compressed gas pump.

### DETAILED DESCRIPTION OF THE PREFERRED EMBODIMENTS

While the description below sets forth several possible embodiments of the system, device or method, it will be appreciated that particular details of the description are for the purpose of illustration only and should not be construed as limiting the present invention. Furthermore, various applications of the inventions, and modifications thereto, which will be apparent to those who are skilled in the art, are also contemplated. For example, while the preferred embodiments are particularly useful in the context of enteral feeding, the systems, devices or methods may be useful in other applications as well, including a wide variety of dispensing applications for flowable substances.

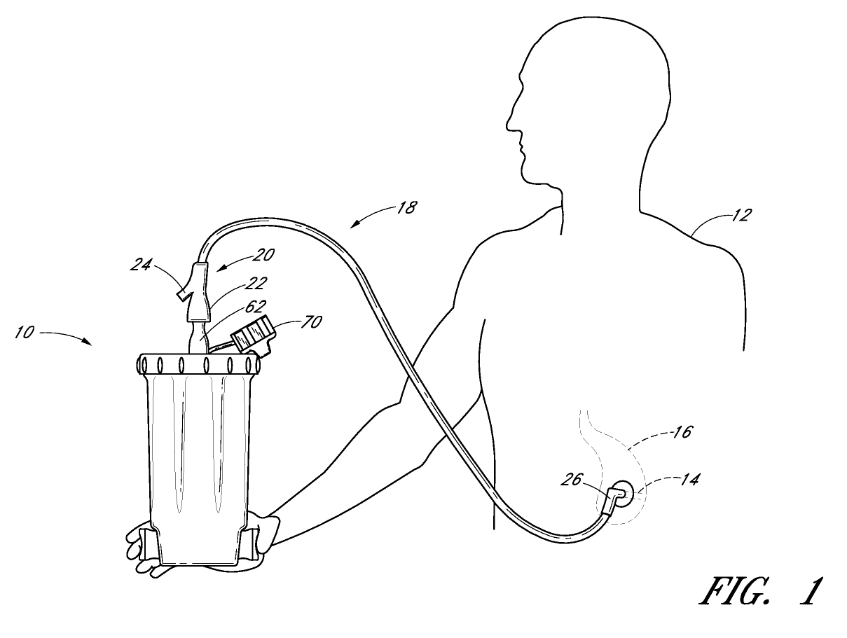

FIG. 1 illustrates an enteral feeding system including an enteral feeding device 10 being utilized to provide a nutritional substance to a user or patient 12. In the illustrated arrangement, the user 12 is self-administering the nutritional substance using the system. In other arrangements, a user of the system may be administering a nutritional substance to another person or to a non-human animal. The term “user” is used in its ordinary meaning throughout the present disclosure and is intended to refer to a person interacting with at least a portion of the system to administer a nutritional substance to himself or herself, or to another person or non-human animal. The term “user” may also refer to a person or non-human animal that receives the nutritional substance through administration by another. The term “patient” usually refers to a person or non-human animal that receives the nutritional substance through administration by another, unless the context of the usage implies otherwise.

The enteral feeding system of FIG. 1 is capable of receiving a nutritional substance, or other flowable substance, and delivering the flowable substance to the user 12. Advantageously, the nutritional substance may be mixed, if necessary, after being added to the feeding device 10. The term “nutritional substance” is user herein in its ordinary meaning and is intended to refer generally to a flowable substance that is suitable for administration to the gastrointestinal (GI) system of a human or non-human animal. Although such a nutritional substance will often contain caloric substances, the term is also intended to cover non-caloric substances as well, such as water, for example, unless otherwise indicated.

In the illustrated arrangement, the user 12 includes an enteral feeding tube 14 that extends from a location external of the body of the user 12 to a location within the GI system of the user 12, such as the stomach 16. Such a feeding tube 14 is often generally referred to as a gastrostomy tube and, in particular, a percutaneous endoscopic gastronomy (PEG). One particular type of gastrostomy tube, which is usually short and does not extend substantially beyond the skin level in an outward direction, is referred to as a gastrostomy button. Although a gastrostomy tube (or button) is illustrated, it will be appreciated that the system 10 may be used with a variety of feeding tubes or other feeding conduits. For example, the system 10 may be used with any type of gastric tube (G-tube), jejunal tube (J-tube) or any other suitable enteral feeding device.

As illustrated in FIG. 1, typically, an intermediate tube or conduit, such as an extension set 18, extends between the enteral feeding device 10 and the feeding tube 14. The extension set 18 includes a connecting hub 20 at a first end, which may include a feeding port 22 and, in some arrangements, a medication port 24. The feeding port 22 preferably is configured to be selectively coupled to the feeding device 10. The medication port 24 is capable of being selectively coupled to a suitable device for delivering a medicinal substance. A second end of the extension set includes a locking adapter 26 that is configured to be selectively coupled to the feeding tube 14. Although an example of a commonly available extension set 18 is illustrated, other suitable intermediate conduit devices may also be used. In addition, in some arrangements, the feeding device 10 may be directly connected to the feeding tube.

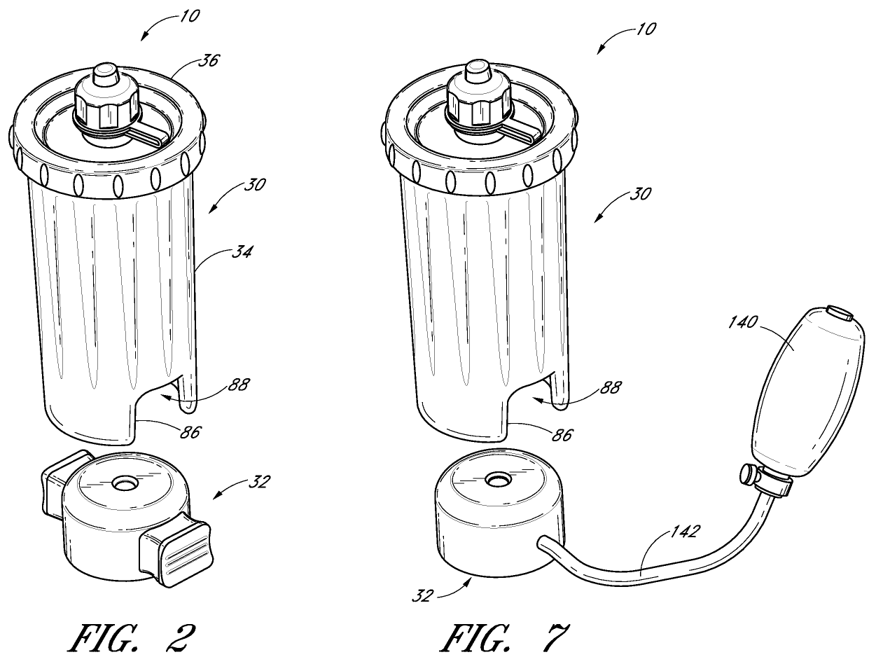

With reference to FIG. 2, the enteral feeding device 10 is illustrated in a partially disassembled state. In particular, the illustrated feeding device 10 includes a storage assembly 30 separated from a pump unit 32. As illustrated in FIGS. 1, 3 and 5, the storage assembly 30 may be selectively coupled with the pump unit 32 such that the pump unit 32 may be utilized to produce a force tending to cause the nutritional substance to be expelled from the storage assembly 30. Although illustrated as separable, in some arrangements, the storage assembly 30 and the pump unit 32 may be integrated with one another. Indeed, in some arrangements, one or more components of the feeding device 10 may define portions of both the storage assembly 30 and the pump unit 32. However, the illustrated separable arrangement advantageously allows the storage assembly 30 to be stored (e.g., refrigerated) or cleaned separately from the pump unit 32 when it may be desirable to do so.

The illustrated storage assembly 30 includes a housing, such as a cup-like container 34, and a closure, such as a lid 36, for example. In the illustrated arrangement, the lid 36 is separable from the container 34 to allow access to the inside of the feeding device 10. In other arrangements, however, the storage assembly 30 may include a permanently sealed housing and closure. In such an arrangement, other features may be present to facilitate access to the interior of the feeding device 10, if such access is desirable. Furthermore, the storage assembly 30 can take on any of a variety of suitable shapes and sizes. Although, in one arrangement, the feeding device 10 is configured to hold enough nutritional substance for a small number of feeding sessions, such as between about 1-5 and, preferably, between about 1-2, for example. In one arrangement, the feeding device 10 is sized to hold a volume of nutritional substance that is satisfactory for a single average feeding session. Such a volume may be between about 250 and 500 milliliters, for example. Thus, in some arrangements, the feeding device 10 may hold up to about 2500 milliliters.

An outer surface of the container 34 is configured with longitudinally-extending channels or grooves 38 that enhance the user's 12 grip on the container 34. The lid 36 includes projections or ridges 40 that also extend in a substantially longitudinal direction. The ridges 40 on the lid 36 enhance the user's 12 grip on the lid 36. Additionally, the ridges 40 inhibit the storage assembly 30 from rolling in the event that it is place on its side.

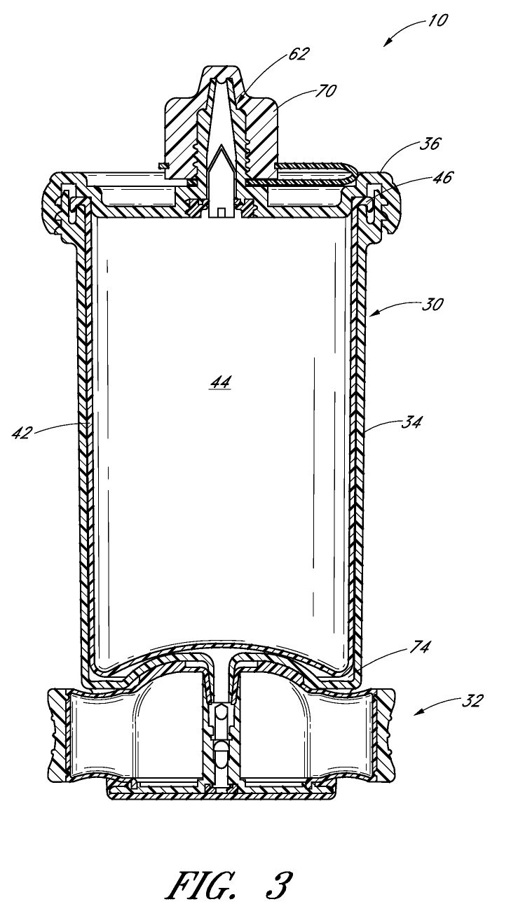

With additional reference to FIGS. 3-5, the storage assembly 30 includes a bladder 42 that at least partially defines a chamber 44 configured to receive the nutritional substance that is to be dispensed from the storage assembly 30. The chamber 44 may be referred to herein as a storage chamber or a dispensing chamber. In the illustrated arrangement, the storage chamber 44 is defined partially by an interior surface of the bladder 42 and partially by an interior surface of the lid 36. In other arrangements, the storage chamber 44 may be defined by additional components, as well. Alternatively, in some arrangements the storage chamber 44 may be defined solely by a bladder insert, which may be disposable.

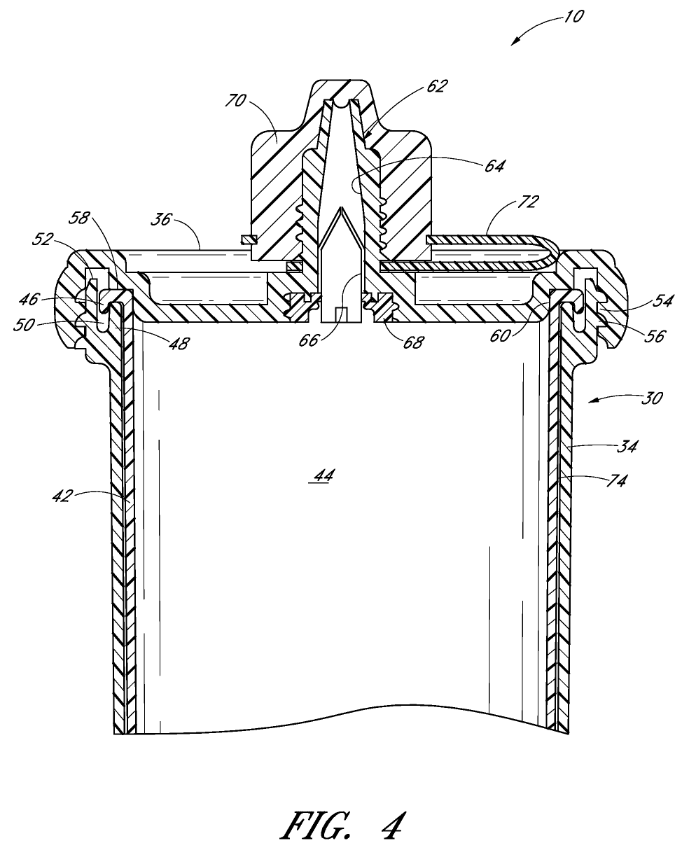

In the illustrated arrangement, the bladder 42 is sized and shaped to generally conform to the interior shape of the container 34. A lower end of the bladder 42 is closed and an upper end of the bladder 42 is open. The upper end portion 46 of the bladder 42 is configured to be captured between the container 34 and the lid 36. In the illustrated arrangement, a seal is created between the lid 36 and the bladder 42 when the lid 36 is sufficiently secured to the container 34. As shown in FIG. 4, the upper end portion 46 of the bladder 42 overlaps an annular projection 48 of the upper end of the container 34 and is received within an annular groove 50 defined between the projection 48 and an annular threaded rim 52 formed at an upper end of the container 34. In some arrangements, the upper end portion 46 of the bladder 42 may be preformed to curve over the projection 48 to ease assembly.

The lid 36 includes internal threads 54 that engage external threads 56 of the threaded rim 52. The lid 36 also includes an annular shoulder 58 that contacts the bladder 42 and compresses the bladder 42 against the annular projection 48 to create a seal between the bladder 42 and the lid 36. Furthermore, preferably, a seal is also created between the bladder 42 and the container 34 such that a chamber is created between an exterior surface of the bladder 42 and the interior surface of the container 34, which is described further below.

The lid 36 also includes an annular surface 60 that extends away from the shoulder 58 towards the interior of the container 34 when the lid 36 is assembled to the container 34. The annular surface 60 preferably is sized to assist in creating or maintaining the seal between the bladder 42 and the lid 36, and the bladder 42 and the container 34. Although such a sealing arrangement is preferred, other suitable arrangements or methods for sealing the bladder 42 to the container 34 or lid 36 may also be used. Furthermore, as discussed above, in some arrangements, a self-contained, sealed bladder may be utilized.

The storage assembly 30 also includes an outlet 62 that permits a nutritional substance to exit the interior of the storage assembly 30. In the illustrated arrangement, the outlet 62 passes through the lid 36. In particular, the outlet 62 is formed by the lid 36. However, in alternative arrangements, the outlet 62 may be formed of a component separate from the lid 36 and may be secured to the lid 36 by any suitable mechanism.

The illustrated outlet 62 defines an outlet passage 64 that extends between a first end, which opens to the storage chamber 44, and a second end, which opens to the exterior of the storage assembly 30. A one-way valve 66 allows fluid to flow through the outlet passage 64 in a direction to exit the storage chamber 44, but inhibits fluid from flowing through the outlet passage 64 in a direction to enter the storage chamber 44. In the illustrated arrangement, the one-way valve 66 is a duckbill-type valve and is held in place by a retention member 68 that is threadably engagable with the lid 36. However, other suitable valve arrangements may also be used, such as an elastomeric slit valve, for example. One such slit valve may be constructed of a silicone material.

Preferably, a cap 70 is provided to selectively cover the outlet 62. The illustrated cap 70 is threadably engagable with the outlet 62; however, in other arrangements, the cap 70 may engage the outlet 62 in a snap-fit arrangement. Alternatively, instead of a cap 70, the outlet may include a screw-type closure, such as a closure similar to those commonly utilized on condiment bottles, for example. The illustrated cap 70 includes a strap 72 that couples the cap 70 to the lid 36 to avoid loss of the cap 70 when it is removed from the outlet 62.

Regardless of the specific construction, preferably, the outlet 62 is configured to be connectable to a feeding tube 14. In the illustrated arrangement, the outlet 62 is configured to be connectable, directly or indirectly, to an intermediate conduit, such as the extension set 18 (FIG. 1). Preferably, a retention force between the extension set 18 and the outlet 62 is sufficient to maintain a connection therebetween in response to pressure within the feeding device 10 and normal or expected forces acting to separate the extension set 18 and outlet 62. Further, preferably the retention force is low enough that the extension set 18 and outlet 62 will separate in response to excessive forces, such as in the event that the feeding device 10 falls or is dropped, or otherwise moves drastically or unexpectedly.

As described above, a chamber 74 is formed between the interior surface of the container 34 and the exterior surface of the bladder 42. The chamber 74 is referred to herein as the pressure chamber. The pressure chamber 74 is configured to receive a pressurizing fluid, preferably a gas, which applies a force tending to collapse the bladder 42 to expel a nutritional substance from the storage chamber 44.

The pressurizing fluid enters the pressure chamber 74 through an inlet 76 in the container 34. The illustrated inlet 76 is formed in a bottom surface of the container 34 and defines an inlet passage 78 that permits the pressurizing fluid to enter the pressure chamber 74. The inlet 76 may be integrally formed with the container 34, as shown, or may be formed by a separate member that is coupled to the container 34.

In the illustrated arrangement, the pump unit 32 can be selectively secured to the inlet 76. As described above, the pump unit 32 is configured to introduce a pressurizing fluid, preferably a gas, into the pressure chamber 74. The illustrated pump unit 32 is a manual pump that pumps air in response to the application of a manual force sufficient to deform a resilient pump bladder, or bellows 80.

The illustrated pump bladder 80 includes at least one, and preferably a pair of input buttons 82 that define finger grip surfaces 84. A user 12 places his or her fingers on the finger grip surfaces 84 and squeezes the input buttons, or push buttons 82 to deform the pump bladder 80, thereby expelling air from the interior of the pump bladder 80 into the pressure chamber 74.

The illustrated container 34 is specifically configured to accommodate the pump unit 32. In particular, the lower portion of the container 34 includes at least one, and preferably two openings, or windows 86 in an outer surface of the container 34. The windows 86 open into an internal cavity 88 formed by a lower surface of the container 34. The cavity 88 is sized and shaped to receive the pump unit 32. Preferably, the height of the cavity 88 is slightly greater than the height of the pump unit 32 such that the lowermost surface of the container 34 can be set on a surface when the pump unit 32 is assembled to the container 34, as illustrated in FIGS. 3 and 5. The openings 86 are configured to allow the push buttons 82 to pass therethrough. Such an arrangement provides functionality, with a simple and pleasing external appearance, and inhibits undesired or unintentional separation of the storage assembly 30 from the pump unit 32. Although the illustrated feeding device 10 places the storage assembly 30 above the pump unit 32, in other arrangements the pump unit 32 may be above the storage assembly 30. In such an arrangement, the outlet 62 from the storage chamber 44 may be located at or near the bottom of the storage assembly 30. Such an arrangement allows gravity to assist in dispensing the nutritional substance from the storage chamber 44. A suitable valve, such as an on-off type valve, or petcock, may be utilized to control fluid flow from the outlet 62. The pump unit 32 may be located at or near the top of the storage assembly 30, such as integrated into the lid 36, for example. The pump unit 32 can be of any suitable arrangement, and may pressurize a pressure chamber 74 or may pressurize an air space within the storage chamber 44.

Refill air is provided to a pump volume space 90 of the pump bladder 80 through a refill passage 92. The refill passage 92 is formed in a base plate 94 that supports a lower end of the pump bladder 80. The shaft 96 extends upwardly from the base plate 94 and, in the illustrated arrangement, is integrally formed with the base plate 94. An upper end of the shaft 96 supports an upper end of the pump bladder 80.

An opening, or port 98, extends radially through the shaft 96 and communicates with the refill passage 92. A one-way valve 100 is positioned within the refill passage 92 between an exterior opening of the refill passage 92 and the port 98. The one-way valve 100 allows refill air to enter the pump volume space 90 from outside the pump unit 32 through the refill passage 92. The one-way valve 100 also inhibits and preferably at least substantially prevents air from escaping through the refill passage 92 when the pump bladder 80 is compressed.

Another one-way valve 102 is positioned in an upper portion 104 of a passage defined by the shaft 96, between the inlet passage 78 and the port 98. The one-way valve 102 permits air to flow from the pump volume space 90 to the pressure chamber 74. The one-way valve 102 also inhibits and preferably at least substantially prevents air from the re-entering the pump volume space 90 from the pressure chamber 74. The illustrated one-way valves 100, 102 are duckbill-type valves; however, other suitable types of valves may also be used.

In operation, the user 12 squeezes the push buttons 82 to expel air from the pump volume space 90 to the pressure chamber 74. The squeezing of the push buttons 82 may be repeated at a user selected magnitude and frequency to pressurize the pressure chamber 74 and cause the nutritional substance to be expelled from the storage chamber 44 at a desired flow rate. Advantageously, such an arrangement provides the ability for the user 12 to select a desired flow rate of the nutritional substance, or feed rate, from a wide variety of possible feed rates. Moreover, the feed rate can be easily, and even substantially, varied throughout the course of a single feeding. As a result, the user 12 can substantially mimic the feed rates, and variation of feed rate, that are achievable through oral feeding. For example, with a peristaltic pump enteral feeding method, a feed rate may be selected from an available range of about 1-500 ml/hr. Furthermore, with such a peristaltic pump, the feed rate typically is constant throughout a feeding session. In contrast, with at least one preferred embodiment of the present enteral feeding system 10, the user 12 may achieve a feed rate of up to about 6000 ml/hr. Accordingly, if desired, a given volume of nutritional substance may be delivered up to about 12 times faster than with existing peristaltic pumps. Moreover, the feed rate during any particular feeding session may be widely varied. As such, the user 12 is better able to mimic the feed rates and rhythms of oral feeding, which may lead to increased satisfaction for the user 12.

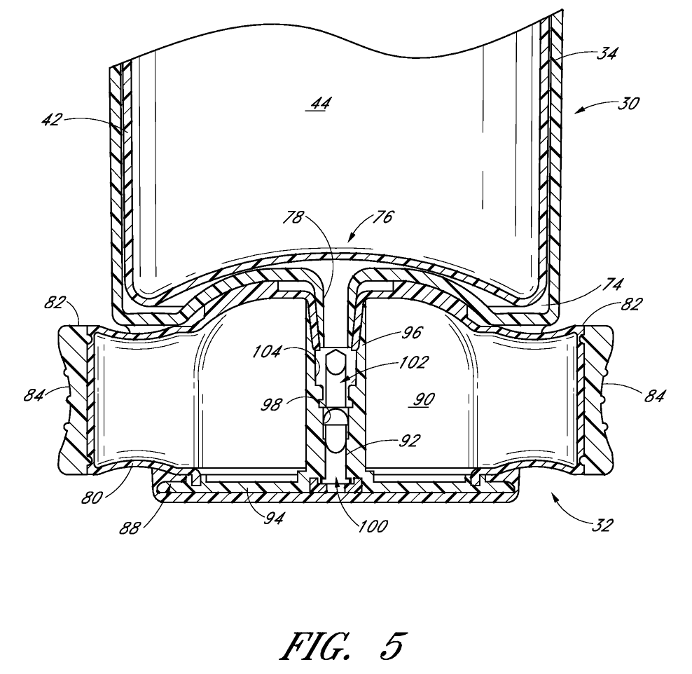

FIG. 6 is a flowchart that provides one example of a process for using the enteral feeding device 10 described above, as well as the additional devices 10 described herein and alternatives and obvious variations of such devices 10. The flowchart of FIG. 6 is described with additional reference to the feeding device 10 and user 12 of FIGS. 1-5. Moreover, the process may include additional activities from those described, or may omit activities described herein, or both.

At block 110, a nutritional substance, or other fluid desired to be delivered, is added to the storage chamber 44. Such a nutritional substance or other fluid may be premixed and ready for consumption. Thus, the nutritional substance can be poured directly into the storage chamber 44.

However, in some cases, it may be desirable to deliver an elemental formula, which is a formula containing fully or partially broken down proteins. Such elemental formulas typically have a short shelf life, such as only a few hours. Advantageously, the embodiments described herein, and obvious variants thereof, are well-suited to permit mixing of a nutritional substance within the storage assembly 30. As an option to block 110, at block 112, a nutritional substance ingredient, such as a powder or concentrate, may be added to the storage chamber 44. At block 113, the ingredient(s) may be stored in the storage chamber 44, if desired. At block 114, water, or another desired liquid, may be added to the storage chamber 44. At block 116, the water (or other liquid) and powder may be mixed, for example, by shaking the storage assembly 30. The option to mix the nutritional substance directly in the storage assembly 30 provides a significant advantage over the existing devices, systems and methods of enteral feeding, which typically use a bag for holding the nutritional substance. As discussed above, syringes are also unsuitable for mixing. When using a syringe, the nutritional substance typically is mixed in one container and then drawn into the syringe. Even if the prior art bag permits the liquid and powder to be added separately to the bag, mixing the liquid and powder within the bag is difficult due to the shape of the interior space of the bag and the lack of rigid walls.

Regardless of the whether the nutritional substance is premixed, or mixed within the storage assembly 30, at block 118 the user 12 prepares the feeding device 10. It should be noted that the preparation of the feeding device 10 does not necessarily occur immediately after the nutritional substance is added to the feeding device 10. Another advantage of the disclosed feeding device 10 is that it is suitable for storing the nutritional substance for later use. The stored substance may be in hydrated or non-hydrated form.

In preparing the feeding device 10, the lid 36 may be secured to the container 34, if necessary. In addition, the storage assembly 30 may be coupled to the pump unit 32 and the extension set 18 (if used) connected to the storage assembly 30. The user 12 may also desire to prime the system to remove a portion or all of the air from the system 10. Because the nutritional substance is delivered to the GI system (as opposed to intravenously, for example), some amount of air can be tolerated. The user 12 may actuate the pump unit 32 to prime they system 10, if desired. Other suitable methods of removing air from the system 10 may also be used. Once primed, the extension set 18 may be connected to the feeding tube 14.

At block 120, the user 12 actuates the pump unit 32. The pump unit 32 described above is manually-actuated and utilizes energy provided by the user 12 to pressurize the pressure chamber 74. However, as discussed above, the pump unit 32 is not necessarily manually-actuated. For example, other types of mechanical, electric or gas pumps, and other suitable pumps, may also be used. Examples of alternative pump units 32 are described below.

At block 122, pressure generated by the actuation of the pump unit 32 at block 120 pressurizes the pressure chamber 74. Pressure in the pressure chamber 74, when sufficient, tends to collapse the bladder 42 and expel the nutritional substance from the storage chamber 44, at block 124. At block 126, the expelled nutritional substance is delivered to the feed tube 14. In the illustrated arrangement, the nutritional substance is delivered to the feed tube 14 via the extension set 18. However, as discussed above, other suitable arrangements may be used.

At block 128, the nutritional substance is received by the user 12. It should be noted that the actuation of the pump unit 32 at block 120, at least initially, may not sufficiently pressurize the pressure chamber 74 to cause the nutritional substance to reach the user 12, especially for new users 12 that are unfamiliar with the feeding device 10. Thus, the actuation of the pump unit 32 at block 120 may be repeated during any of the activities at the blocks that follow (e.g., 122-130). Once familiar with the feeding device 10, it is likely that the user 12 will actuate the pump unit 32 sufficiently to cause delivery of at least some portion of the nutritional substance within the storage chamber 44.

At block 130, the user 12 monitors the delivery feed rate and determines if or when to actuate the pump unit 32 to maintain or increase the delivery feed rate of the nutritional substance. Monitoring of the delivery feed rate may be accomplished by the sensory or physiological response resulting from the delivery of the nutritional substance to the GI system. In addition, or in the alternative, monitoring may be done visually, or by any other suitable method. “Monitoring” of the delivery feed rate may be as simple as determining if any nutritional substance is being delivered at any particular point in time. That is, it is not necessary that the user 12 know the actual feed rate. If desired, however, suitable flow meters may be used to determine the feed rate and/or flow restrictors may be used to limit the maximum feed rate. As noted above, the monitoring (and actuation of the pump unit 32) may be done by the recipient of the nutritional substance, by one or more helpers, or any combination thereof.

If it is determined to actuate the pump unit 32 to maintain or increase the delivery feed rate, the pump unit 32 is actuated at block 120. The process then repeats blocks 122-130, substantially as described above. This process loop may be continued until the supply of the nutritional substance within the storage chamber 44 is exhausted, or at any time prior. Furthermore, at any point within the feeding process, the user 12 can stop the flow of the nutritional substance, such as through a clamp on the extension set 18, for example. The flow can then be restarted when desired. If sufficient pressure remains within the pressure chamber 74, delivery of the nutritional substance may continue. Further, the feeding device 10 may employ other flow control devices, such as a fixed or variable flow regulator at any suitable location within the system. Advantageously, the use of a flow restrictor can increase the duration of delivery of the nutritional substance for a given pressure in the pressure chamber 74.

Preferably, the feeding device 10 and other parts of the system, such as the extension set 18, are cleaned and prepared for a further use. If desired, the feeding device 10 may be used, in a manner similar to that described above, to flush the feed tube 14 and/or extension set 18, or other system components, with water or another suitable cleaning or flushing agent. As noted above, particular arrangement of the storage assembly 30 promotes easy cleaning. The bladder 42 may be easily removed from the container 34 and cleaned for reuse. The relatively simple shape of the bladder 42, with the large open end, makes the bladder 42 easy to clean, at least in comparison with prior art bags. Alternatively, the bladder 42 may be discarded and replaced with a new bladder 42 for the next use.

In one alternative arrangement, the feeding device 10 and, in particular, the storage assembly 30, may be configured for use with a bladder insert. The bladder insert preferably is designed for a single use, but may be capable of being used several times before being replaced. The bladder insert preferably would include a deformable bladder body and a closure or lid. The lid would incorporate an outlet nozzle capable of connection to a feeding tube 14, directly or via another component, such as an extension set 18. The bladder and lid could be separate components that are held together to create a seal by the storage assembly 30. For example, the bladder and lid could be captured between the container 34 and lid 36, much like the bladder 42 described above. In such an arrangement, the lid 36 of the storage assembly could include a central opening to allow the outlet nozzle of the bladder insert to be placed therethrough. Alternatively, the bladder insert may be a single component that is simply inserted into the feeding device 10. Such a bladder insert may be prefilled with a nutritional substance.

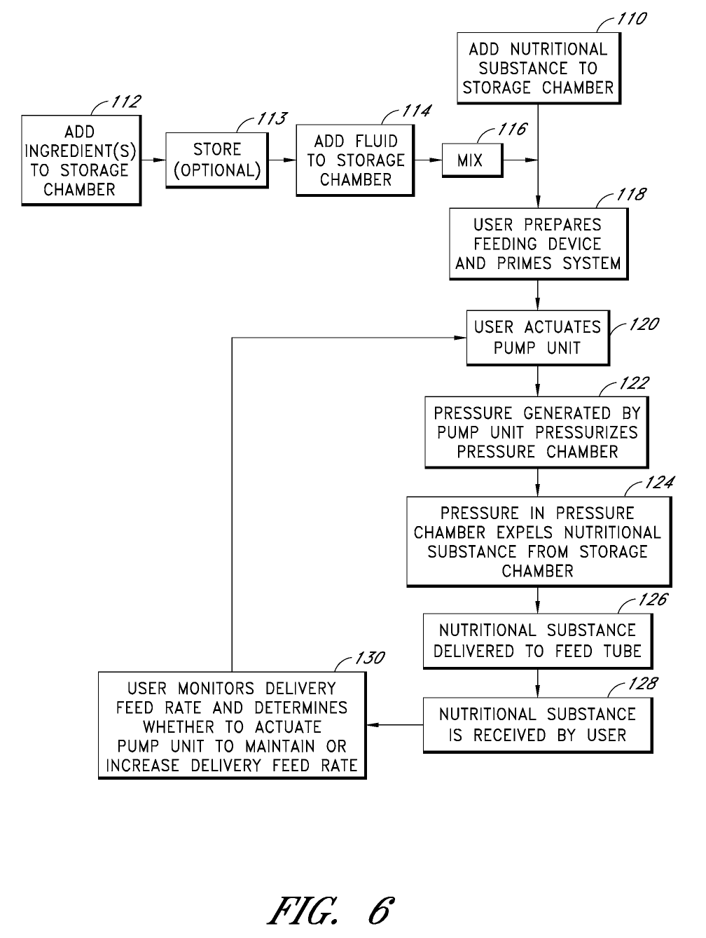

FIGS. 7-9 illustrate alternative feeding devices, which are similar to the above-described device 10 in most respects, unless otherwise noted below. Accordingly, the reference numbers used above are repeated below to describe the same or similar components. Preferably, the storage assemblies 30 of FIGS. 7-9 are substantially similar to the storage assembly 30 described in detail above, or to the above-described alternatives. However, the pump units 32 of FIGS. 7-9 vary from the pump unit 32 described above.

The pump unit 32 of FIG. 7 is also manually-actuated. The pump unit 32 includes a hand bulb 140 that can be squeezed by the user 12. Squeezing of the hand bulb 140 causes a pressurizing fluid, preferably air, to be delivered to the pressure chamber 74 of the storage assembly 30. The pump unit 32 may include any valve(s) necessary to ensure that air is delivered to the pressure chamber 74 when the hand bulb 140 is squeezed and to allow the hand bulb 140 to refill with air when it is released.

A conduit, such as a tube 142, couples the hand bulb 140 to the body of the pump unit 32, which preferably is received within a cavity 88 of the storage assembly 30, as described above. The tube 142 may be passed through one of the openings 86 so that the storage assembly 30 can sit upright on a supporting surface. The opening 86 may be sized and/or shaped specifically to accommodate the tube 142.

Such manually-actuated pumps are capable of pressurizing the pressure chamber 74 using energy originating from the user 12. As a result, the feeding device 10 may be used nearly anywhere without the need for an external source of power. In addition, the manually-actuated pumps are typically lightweight to enhance portability and relatively simple to use. The manually-actuated pumps also provide the user 12 with a great deal of control over the delivery feed rate and the overall rhythm of the delivery. However, in certain situations, or for certain applications, an external source of power may be desirable.

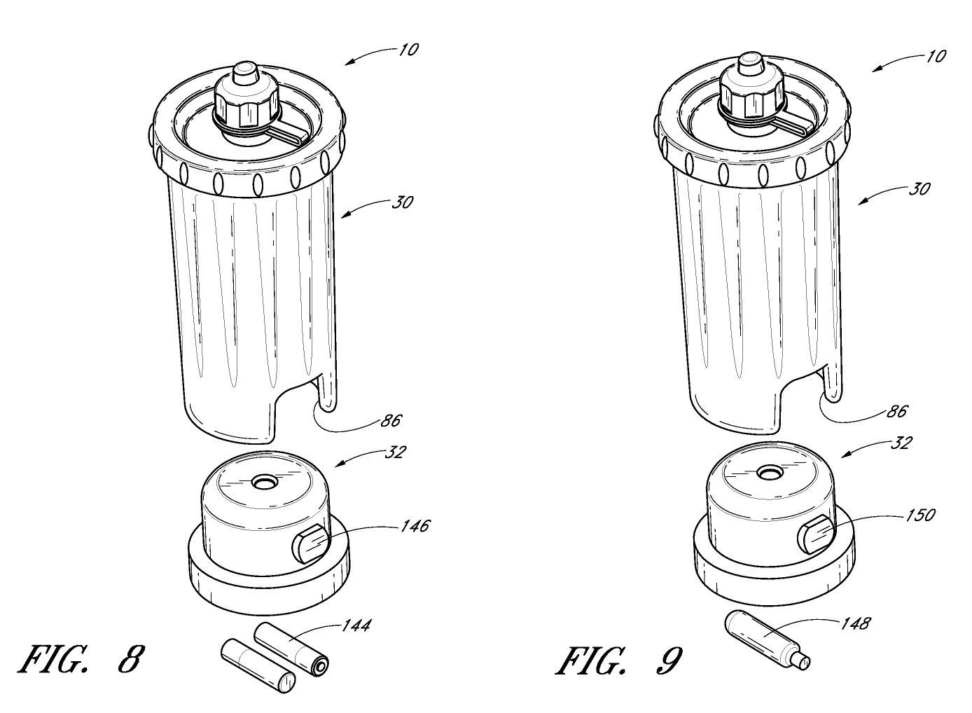

FIG. 8 illustrates a feeding device 10 in which the pump unit 32 utilizes an electric pump. The illustrated electric pump is powered by one or more batteries 144; however, AC powered or plug-in type pumps may also be used. The electric pump may be of any suitable design that is capable of delivering a pressurizing fluid, such as air, to the pressure chamber 74 at a suitable rate. The pump unit 32 includes a user switch, or actuation button 146. The actuation button 146 may be a toggle-type switch that turns the electric pump on and off with successive actuations of the button 146. Alternatively, the button 146 may simply turn the electric pump on when the button 146 is depressed and turn the electric pump off when the button 146 is released. The button 146 may be positioned on the pump unit 32 to be accessible through the opening 86 of the storage assembly 30. As will be apparent to one of skill in the art, other types of switches, controls, actuators or user inputs may be utilized to control the electric pump as desired. For example, a control that permits variable flow rates of the electric pump to be achieved may be desirable. In some arrangements, a controller, such as a microprocessor, may be incorporated into the pump unit 32 such that the pump unit 32 may be programmed to carry out a feeding protocol. For example, such a pump unit 32 could be programmed for extended term feeding, such as overnight feedings in a manner similar to programmable peristaltic pumps that are currently available.

FIG. 9 illustrates a feeding device 10 in which the pump unit 32 utilizes a compressed gas-type pump. In the illustrated arrangement, a compressed gas cartridge 148 is employed to deliver a pressurizing gas to the pressure chamber 74. Preferably, the cartridge 148 is the same or similar to commonly available CO2 cartridges (threaded or unthreaded) used, for example, with air rifles and various inflation devices. A button 150, or other suitable user control, may be configured to control a valve that, in turn, controls the release of gas from the cartridge 148. The button 150 may be positioned on the pump unit 32 to be accessible through the opening 86 of the storage assembly 30.

Although the above-described mechanisms for expelling the nutritional substance from the storage chamber 44 provide desirable advantages over one or more of the existing enteral feeding systems, other suitable devices may also be employed. For example, a driven piston arrangement may be used to expel the nutritional substance from the storage chamber 44. The piston may be a separate component that is movable within the storage assembly 30 to act on the bladder 42 (or directly on the nutritional substance), or may be formed by a portion of the bladder 42 itself, such as a rigid member or wall provided on the bladder 42, for example. The piston may be driven by fluid pressure, by a suitable mechanical mechanism (such as a spring), or by other suitable arrangements.

Another alternative arrangement is to configure at least a portion of the container 34 to be resiliently deflectable. Deflection of the container 34 could be used to pressurize the pressure chamber 74, which could be separated from a pump chamber, which is reduced in volume due to deflection of the container 34, by a one-way valve. Alternatively, a pump bladder may be incorporated into, or integrated with, the container 34 and be separated from the pressure chamber 74 by a one-way valve. The pump bladder may be similar to the pump bladder 80 of the pump unit 32 described in connection with FIGS. 1-5. Alternatively, the pump bladder may be similar to a pump button used to inflate athletic shoes or air casts, for example.

In one arrangement, the pump comprises a resilient diaphragm that is held in place relative to the storage assembly 30 by a retaining member, such as a retaining ring. For example, the diaphragm may be positioned within the cavity 88 of the storage assembly 30 and function to pressurize the pressure chamber 74 when the diaphragm is depressed. A one-way valve may be positioned in any suitable location to allow refill of air when the diaphragm resiliently returns to its normal orientation.

One of skill in the art will recognize that the above-described arrangements are only examples of possible implementations of the present enteral feeding systems, devices and methods. Moreover, it will be appreciated that a variety of suitable materials and manufacturing processes may be used to produce the various components of the system, such as those specific components described above. For example, the containers 34 and lids 36 may be constructed of relatively rigid plastic materials suitable for use with food products. Some examples of suitable materials include ABS plastic, PC/ABS plastic, polyethylene, and polypropylene.

The bladder 42 and pump bladder 80 may be constructed of relatively flexible materials. The bladder 42 preferably is readily collapsible to enhance the efficiency of the delivery of the nutritional substance in response to pressure within the pressure chamber 74. However, the bladder 42 preferably is also relatively tough, to avoid tearing, and is suitable for contact with food products. More preferably, the bladder 42 is constructed from a material that is suitable for use with those that suffer from severe allergies. Some examples of suitable materials for the bladder 42 include suitable elastomeric materials, such as silicon and low density polyethylene. The type of material and/or thickness of the material for the bladder 42 may also be selected based on whether the bladder 42 is intended to be reused, or if the bladder 42 is intended to be disposable. The pump bladder 80 preferably is constructed from a material that is collapsible in response to pressure (such as manual pressure exerted by the user 12, but is also resilient enough to return to its original shape when the pressure is released to thereby enhance the pumping efficiency. Some examples of suitable materials for the pump bladder 80 include silicon and rubber.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In particular, while the present enteral feeding system has been described in the context of particularly preferred embodiments, the skilled artisan will appreciate, in view of the present disclosure, that certain advantages, features and aspects of the system may be realized in a variety of other applications, such as the delivery of other types of flowable substances. Additionally, it is contemplated that various aspects and features of the invention described can be practiced separately, combined together, or substituted for one another, and that a variety of combination and subcombinations of the features and aspects can be made and still fall within the scope of the invention. Thus, it is intended that the scope of the present invention herein disclosed should not be limited by the particular disclosed embodiments described above, but should be determined only by a fair reading of the claims.

What is claimed is:

1. A method of delivering a flowable nutritional substance to a feeding tube, comprising:
applying a manual force to an input of a manual pump;
pressurizing a pressure chamber in response to energy originating from the application of the manual force to the input of the manual pump;
expelling the flowable nutritional substance from a storage chamber in response to the pressure in the pressure chamber; and
delivering the expelled flowable nutritional substance to the feeding tube.

2. The method of claim 1, wherein the manual force is applied at a user-selected magnitude and frequency such that a rate of the delivery of the flowable nutritional substance generally corresponds to the user's desired rate of consumption.

3. The method of claim 1, wherein at least a portion of the application of the manual force occurs during the delivery of the expelled nutritional substance.

4. The method of claim 1, further comprising inserting a bladder into a container to at least partially define the pressure chamber and the storage chamber.

5. The method of claim 4, further comprising securing a lid to the container to capture an open upper end of the bladder between the container and the lid whereby the pressure chamber is created between the container and the bladder and the storage chamber is created between the bladder and the lid.

6. The method of claim 5, further comprising adding the nutritional substance to the storage chamber prior to securing the lid to the container.

7. The method of claim 1, wherein the manual pump is incorporated in a pump unit that is capable of being coupled to the container, the method further comprising coupling the pump unit and the container prior to applying the manual force to the input of the manual pump.

8. The method of claim 1, wherein the flowable nutritional substance is provided to the storage chamber as a plurality of ingredients that are subsequently mixed with one another in the storage chamber.

9. An assembly for delivering a flowable nutritional substance to a feeding tube, comprising:
a container having an open end and a substantially closed end, said closed end incorporating a one-way valve that permits a pressurizing fluid to enter said container;
a bladder having an open end, said bladder positionable within said container with said open end of said bladder proximate said open end of said container;
a lid that is securable to said container, said lid and said container configured to capture said open end of said bladder between said lid and said container to create a pressure chamber between said container and an outer surface of said bladder, and to create a storage chamber between said lid and an inner surface of said bladder, said storage chamber configured to receive said nutritional substance;
a pump configured to pump said pressurizing fluid into said pressure chamber to cause said nutritional substance to be expelled from said storage chamber.

10. The assembly of claim 9, further comprising an outlet in said lid through which said nutritional substance is expelled from said storage chamber.

11. The assembly of claim 9, wherein said pump is separable from said container.

12. The assembly of claim 9, wherein said pump is a mechanical pump comprising a deformable pump member that is deformable in response to a user input to pump said pressurizing fluid.

13. The assembly of claim 12, wherein said deformable pump member comprises a pair of push buttons.

14. The assembly of claim 12, wherein said deformable pump member comprises a hand bulb.

15. The assembly of claim 9, wherein said pump is an electric pump.

16. The assembly of claim 9, wherein said pump is a compressed gas pump.

17. An assembly for delivering a flowable nutritional substance to a feeding tube, comprising:
a storage assembly for said nutritional substance, comprising:
a container incorporating a valve that permits a pressurizing fluid to enter said container;
a lid that closes said container;
a bladder positionable within said container, said bladder at least partially defining a storage chamber for said nutritional substance;
wherein a pressure chamber is defined between said container and an outer surface of said bladder;
a pump unit comprising a pump, wherein said pump unit is separate from said storage assembly, said pump unit configured to receive said container and to pump said pressurizing fluid through said valve into said pressure chamber to cause said nutritional substance to be expelled from said storage chamber.

18. The assembly of claim 17, wherein said lid is removable from said container.

19. The assembly of claim 18, wherein an open end of said bladder is captured between said lid and said container to create a seal between said lid and said bladder.

20. The assembly of claim 19, further comprising an outlet in said lid through which said nutritional substance is expelled from said storage chamber.

21. The assembly of claim 17, wherein said pump is a mechanical pump comprising a deformable pump member that is deformable in response to a user input to pump said pressurizing fluid.

22. The assembly of claim 21, wherein said deformable pump member comprises a pair of push buttons.

23. The assembly of claim 21, wherein said deformable pump member comprises a hand bulb.

24. The assembly of claim 17, wherein said pump is an electric pump.

25. The assembly of claim 17, wherein said pump is a compressed gas pump.
